(19) United States
(12) Patent Application Publication
(10) Pub. No.: US 2024/0341782 A1
(43) Pub. Date: Oct. 17, 2024

(54) THROMBECTOMY SYSTEM WITH LINEAR MAGNETIC ENCODER TRAVEL SENSING

(71) Applicant: BOSTON SCIENTIFIC SCIMED, INC., Maple Grove, MN (US)

(72) Inventors: Lee Kantor, Rogers, MN (US); Matthew Protas, Blaine, MN (US); Jason W. Sprain, Shoreview, MN (US)

(73) Assignee: BOSTON SCIENTIFIC SCIMED, INC., Maple Grove, MN (US)

(21) Appl. No.: 18/631,279

(22) Filed: Apr. 10, 2024

Related U.S. Application Data
(60) Provisional application No. 63/458,494, filed on Apr. 11, 2023.

(51) Int. Cl. A61B 17/22; A61B 17/00; A61B 90/00

(52) U.S. Cl. CPC A61B 17/22; A61B 17/00234; A61B 90/06; A61B 2017/00199; A61B 2017/00238; A61B 2017/00398; A61B 2017/22079; A61B 2090/061; A61B 2217/005; A61B 2217/007

(57) ABSTRACT

Thrombectomy systems including a stroke length detection assembly. An illustrative drive unit for a thrombectomy system may comprise one or more panels enclosing an internal structure of the drive unit, a vertically oriented reciprocating linear actuator assembly extending between one or more support structures in an upper region of the drive unit and including a reciprocating linear actuator secured to a mounting plate and an actuator shaft, a bracket including a first portion extending generally parallel to a longitudinal axis of the actuator shaft and a second portion extending generally orthogonal to the longitudinal axis of the actuator shaft, the second portion coupled to the actuator shaft, a magnetic strip affixed to a surface of the first portion of the bracket, and a magnetic sensor positioned adjacent to a first end region of the bracket. The magnetic sensor may be configured to determine a relative position of the actuator shaft.

### CROSS-REFERENCE TO RELATED APPLICATIONS

This application claims the benefit of U.S. Provisional Patent Application Ser. No. 63/458,494, filed Apr. 11, 2023, which is incorporated herein by reference.

### TECHNICAL FIELD

The disclosure is directed to thrombectomy systems. More particularly, the disclosure is directed to a thrombectomy system with linear magnetic encoder traveling sensing for determining a position of an actuator.

### BACKGROUND

Thrombectomy is a procedure for removing thrombus from the vasculature of a patient. Mechanical and fluid-based systems can be used to remove thrombus. With fluid-based systems, an infusion fluid may be infused to a treatment area of a vessel with a catheter to dislodge the thrombus. In some instances, an effluent (e.g., the infusion fluid and/or blood) including the dislodged thrombus may be extracted from the vessel through the catheter. Of the known thrombectomy systems and methods, there is an ongoing need to provide alternative configurations of thrombectomy catheters and systems, as well as methods of operating such thrombectomy systems.

### SUMMARY

This disclosure provides design, material, manufacturing method, and use alternatives for medical devices.

In a first example, a drive unit for a thrombectomy system may comprise one or more panels enclosing an internal structure of the drive unit, a vertically oriented reciprocating linear actuator assembly extending between one or more support structures in an upper region of the drive unit, the vertically oriented reciprocating linear actuator assembly including an actuator shaft, a bracket coupled to the actuator shaft and moveable therewith, the bracket including a first portion extending generally parallel to a longitudinal axis of the actuator shaft, a magnetic strip affixed to a surface of the first portion of the bracket, and a magnetic sensor positioned adjacent to a first end region of the bracket. The magnetic sensor may be configured to determine a relative position of the actuator shaft.

Alternatively or additionally to any of the examples above, in another example, the magnetic sensor may be secured within a housing secured to an upper surface of a mounting plate.

Alternatively or additionally to any of the examples above, in another example, the magnetic sensor may be mounted to a printed circuit board.

Alternatively or additionally to any of the examples above, in another example, the first portion of the bracket may be configured to extend through an opening in a mounting plate.

Alternatively or additionally to any of the examples above, in another example, the magnetic strip may be positioned between the bracket and the magnetic sensor.

Alternatively or additionally to any of the examples above, in another example, the magnetic strip may include a plurality of north and south pole pairs.

Alternatively or additionally to any of the examples above, in another example, the north and south pole pairs may extend along a length of the magnetic strip.

Alternatively or additionally to any of the examples above, in another example, the magnetic sensor may comprise a system-on-a-chip.

Alternatively or additionally to any of the examples above, in another example, the magnetic sensor may be configured to output an “A” signal and a “B” signal.

Alternatively or additionally to any of the examples above, in another example, if the “A” signal rises from 0 to 1 while the “B” signal is 0, the actuator shaft may be moving in a first direction.

Alternatively or additionally to any of the examples above, in another example, if the “B” signal rises from 0 to 1 while the “A” signal is 0, the actuator shaft may be moving in a second direction opposite the first direction.

Alternatively or additionally to any of the examples above, in another example, if the actuator shaft is moving in the first direction, the magnetic sensor may increment a count of rising edges and if the actuator shaft is moving in the second direction, the magnetic sensor may decrement the count of rising edges.

Alternatively or additionally to any of the examples above, in another example, the count of rising edges may be directly correlated to distance traveled by the actuator shaft.

Alternatively or additionally to any of the examples above, in another example, the magnetic strip may have a length approximately equal to or greater than a stroke length of the actuator shaft.

Alternatively or additionally to any of the examples above, in another example, the magnetic strip may be linearly displaced in direct proportion to linear movement of the actuator shaft.

In another example, a drive unit for a thrombectomy system may comprise one or more panels enclosing an internal structure of the drive unit, a vertically oriented reciprocating linear actuator assembly extending between one or more support structures in an upper region of the drive unit, the vertically oriented reciprocating linear actuator assembly including an actuator shaft, a housing, a magnetic sensor secured within the housing, a bracket coupled to the actuator shaft and moveable direct proportion therewith, the bracket including a first portion extending generally parallel to a longitudinal axis of the actuator shaft, and a magnetic strip including a plurality of north and south pole pairs affixed to a surface of the first portion of the bracket between the bracket and the magnetic sensor. The magnetic sensor may be configured to increment or decrement an incremental pulse count as the magnetic strip moves with the actuator shaft and to determine a distance of travel of the actuator shaft relative to a mechanical zero position based on the incremental pulse count.

Alternatively or additionally to any of the examples above, in another example, the magnetic sensor may be configured to output an “A” incremental pulse signal and a “B” incremental pulse signal.

Alternatively or additionally to any of the examples above, in another example, if the actuator shaft is moving in a first direction, the magnetic sensor may increment the incremental pulse count and if the actuator shaft is moving in a second direction opposite the first direction, the magnetic sensor may decrement the incremental pulse count.

Alternatively or additionally to any of the examples above, in another example, the magnetic strip may be spaced 0.6 millimeters or less from magnetic sensor.

In another example, a method for determining a distance of travel of an actuator shaft of a drive unit for a thrombectomy system may comprise identifying a mechanical zero position of an actuator shaft relative to a magnetic strip, incrementing an incremental pulse count as the magnetic strip moves with the actuator shaft in a first direction, decrementing the incremental pulse count as the magnetic strip moves with the actuator shaft in a second direction opposite the first direction, determining a distance and direction traveled by the actuator shaft based on the incremental pulse count, and outputting the distance and/or direction to a user interface.

The above summary of some example embodiments is not intended to describe each disclosed embodiment or every implementation of the disclosure.

### BRIEF DESCRIPTION OF THE DRAWINGS

The disclosure may be more completely understood in consideration of the following detailed description of various embodiments in connection with the accompanying drawings, in which:

FIG. 1 is a perspective view of an illustrative thrombectomy system;

FIG. 2 is a partially exploded perspective view of the pump, the bubble trap, the connection manifold assembly, and an associated fixture of the pump/catheter assembly for use in the thrombectomy system of FIG. 1;

FIG. 3 is a partially exploded side view of the pump, the bubble trap, the connection manifold assembly, and associated fixture of the pump/catheter assembly for use in the thrombectomy system of FIG. 1;

FIG. 4 is a perspective view of an upper section of the drive unit where the panels and other exterior components have been removed to reveal other components residing in the drive unit;

FIG. 5 is a front view of the illustrative reciprocating linear actuator assembly;

FIG. 6 is an enlarged partial front perspective view of the reciprocating linear actuator assembly of FIG. 5 with a mounting bracket removed;

FIG. 7 is an enlarged partial rear perspective view of the reciprocating linear actuator assembly of FIG. 5;

FIG. 8 is an enlarged partial front perspective view of the reciprocating linear actuator assembly of FIG. 5 with a mounting bracket removed and the actuator shaft axially displaced or raised;

FIG. 9 illustrates a front view of a portion of the magnetic strip adjacent to the magnetic sensor and a plurality of electrical outputs from the magnetic sensor; and

FIG. 10 is a schematic graph illustrating incremental pulse outputs as the magnetic strip moves relative to the magnetic sensor.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

All numeric values are herein assumed to be modified by the term “about”, whether or not explicitly indicated. The term “about” generally refers to a range of numbers that one of skill in the art would consider equivalent to the recited value (e.g., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

Thrombectomy catheters and systems may be used to remove thrombus, plaques, lesions, clots, etc. from veins or arteries. The control console or drive unit of a thrombectomy system may include a linear actuator for driving a pump to supply high-pressure saline to the thrombectomy catheter. Some drive units may utilize fixed location sensors to determine a stroke length of the actuator which may limit the system to a single stroke length detection. Disclosed herein is a stroke length detection system which allows for pump systems having different stroke lengths to be used with a same drive unit.

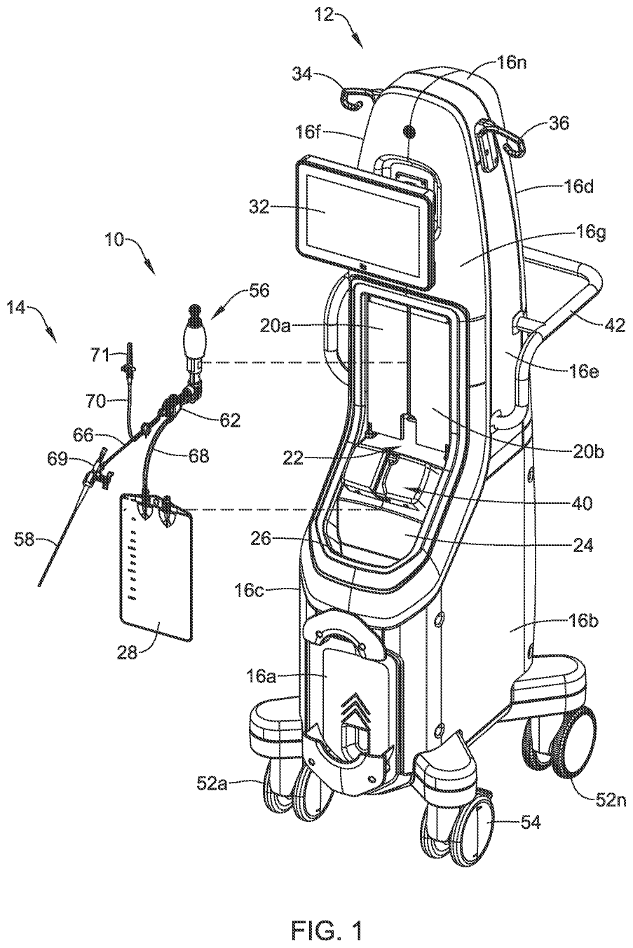

FIG. 1 is a perspective view of an illustrative thrombectomy system 10. The thrombectomy system 10 may include a control console or drive unit 12 and a pump/catheter assembly 14. In some instances, the pump/catheter assembly 14 may be a single use device in which a new pump/catheter assembly 14 may be used with the drive unit 12 for each medical procedure. Shown on the drive unit 12 are a plurality of removable panels 16a-16n about and along the drive unit 12 enclosing the internal structure of the drive unit 12. An illustrative drive unit 12 and pump/catheter assembly 14 are described in commonly assigned U.S. Pat. No. 7,935,077, titled THROMBECTOMY CATHETER DEPLOYMENT SYSTEM, the disclosure of which is hereby incorporated by reference. Centrally located in the drive unit 12 and aligned to the lower region of the panel 16g may be automatically opening loading bay door assemblies 20a, 20b which open to expose the interior of the drive unit 12 to provide access to a carriage assembly 22. The carriage assembly 22, which may accommodate components of the pump/catheter assembly 14 is shown accessible via opening the closed-door assemblies 20a, 20b. The drive unit 12 may include a catch basin for collecting fluid leakage from the components of the pump/catheter assembly 14. For example, a drip tray 24 is shown located on the front of the drive unit 12 extending from below the carriage assembly 22 toward the panel 16a. Other configurations of catch basins are also contemplated. The drip tray 24 and a receptacle 26 may collectively support and accommodate an effluent collection bag, such as effluent collection bag 28 of the pump/catheter assembly 14. In other instances, the drive unit 12 may include a different structure, such as a hook for hanging the effluent collection bag 28 from, or a shelf for setting the effluent collection bag 28 on. The effluent waste tube 68 may also be positioned in the roller pump 40 between the tube guides with the effluent collection bag 28 connected to the effluent waste tube 68. The effluent collection bag 28 may be suitably positioned for collecting effluent during the medical procedure. Pump rollers (not shown) of the roller pump 40 may rotatably engage the effluent waste tube 68 to control effluent fluid flow through the effluent waste tube 68 to the effluent collection bag 28.

In instances where the carriage assembly 22 is movable, a carriage assembly activation switch (not explicitly shown) may be provided with the drive unit 12, such as located on panel 16g, to selectively position the carriage assembly 22 inwardly or outwardly. In other instances, the carriage assembly 22 may be positioned using a user interface 32. A user interface or control panel 32, including memory and/or processing capabilities, may be provided with the drive unit 12, such as located at the upper region of the drive unit 12 between the upper regions of the upper side panels 16e and 16f. The user interface 32 may be a guided user interface (GUI) including a touch screen display to allow a user to provide input to the user interface 32 and view information on a same display screen. However, this is not required. In other instances, the user input may be separate from the display screen. Saline bag hooks 34 and 36 may extend through the panels 16e and 16f to hang saline bags therefrom. The drive unit 12 may include a handle 42 as well as a plurality of wheels 52a-52n and brake pedals 54 for wheel lockage to assist in maneuvering the drive unit 12 by medical personnel.

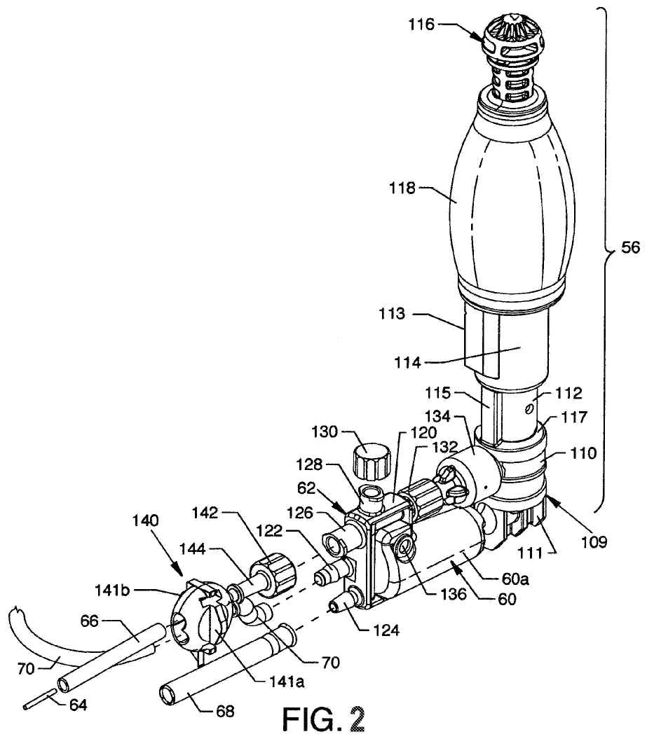

The pump/catheter assembly 14, which may be a disposable single-use device, is shown unattached from the drive unit 12. The pump/catheter assembly 14 includes a pump 56 and a thrombectomy catheter 58. During use, a portion of the pump/catheter assembly 14 may be secured within a portion of the drive unit 12. Other components included in the pump/catheter assembly 14 may include a bubble trap 60 attached to the pump 56, a connection manifold assembly 62 connected to the bubble trap 60, an effluent return tube 66 connected between the connection manifold assembly 62 and the thrombectomy catheter 58, a high-pressure fluid supply tube 64 (see, for example, FIG. 2) attached between the output of the pump 56 and the thrombectomy catheter 58 which may be coaxially arranged inside the effluent return tube 66, a transition fixture 69 between the distal end of the effluent return tube 66 and the proximal end of the thrombectomy catheter 58, an effluent waste tube 68 connecting the effluent collection bag 28 to the connection manifold assembly 62, and a fluid supply tube 70 having a bag spike 71 connecting a fluid supply bag 72 (e.g., a saline bag) to the connection manifold assembly 62. The fluid supply tube 70 may be in fluid communication with the interior of the bubble trap 60 to provide fluid from the fluid supply bag 72 to the pump 56 and then to the thrombectomy catheter 58 through the high-pressure fluid supply tube 64.

FIG. 2 is a partially exploded perspective view of several components of the pump/catheter assembly 14 generally including the pump 56, the bubble trap 60, the connection manifold assembly 62, and a fixture 140. The pump 56 centers about a tubular body 112. Components are located about the lower region of the tubular body 112 and include a base 109 having an upper portion 110 and a lower portion 111 both positioned about the lower region of the tubular body 112. An annular surface 117 is included at the top of the upper portion 110 of the base 109 for intimate contact with capture tabs of the carriage assembly 22 to contain the pump 56 within the carriage assembly 22. A top body 114, is positioned about the upper region of the tubular body 112. The base 109 and the top body 114, as well as a connecting panel 115, may be molded or otherwise suitably constructed to encompass the greater part of the tubular body 112, for example. A data plate 113 may also be included on the top body 114 for the inclusion of a barcode, an RFID tag, or other informational displays to determine operational parameters of the device. The pump 56 may include a hemispherically-shaped pump piston head 116 having a flexible boot 118 connected to and extending between the top body 114 and the pump piston head 116.

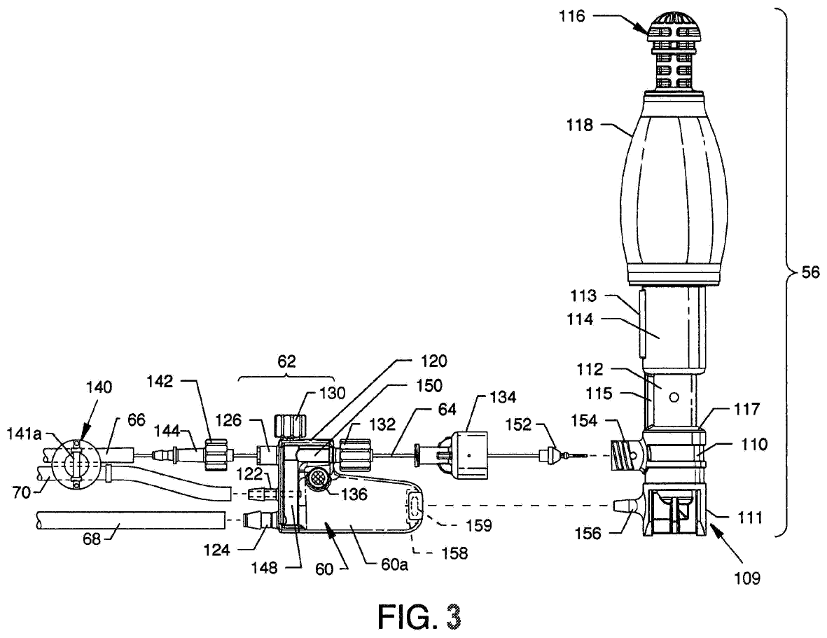

In some instances, the geometrically configured lower portion 111 of the base 109 may serve as a mount for one end of the bubble trap 60 (see, for example, FIG. 3). The connection manifold assembly 62 may be secured directly to the other end of the bubble trap 60 and in some instances may include a bracket 120 to which is attached a vertically oriented tubular manifold 148 having a plurality of ports attached or formed therethrough including a fluid (e.g., saline) inlet port 122, an effluent outlet port 124, a Luer style effluent return port 126, and/or an auxiliary port 128 and cap 130. Also shown are connectors 132 and 134 connectingly extending between the connection manifold assembly 62 and the upper portion 110 of the base 109.

The bubble trap 60 may include mating halves of which one mating half 60a is shown. A hydrophobic filter 136 may be included at the upper forward region of the bubble trap half 60a. Another hydrophobic filter may be included on the second bubble trap half (not explicitly shown) which opposes the hydrophobic filter 136 on the bubble trap half 60a.

The fixture 140, and components associated therewith, assists in support and connection of the effluent return tube 66 to the effluent return port 126 by a connector 142 combined continuously with a connection tube 144, and also assists in support, passage and connection of the fluid supply tube 70 with the fluid inlet port 122. The fixture 140 may include outwardly extending vertically aligned and opposed tabs 141a and 141b which prevent the fixture 140 and associated effluent return tube 66 containing the high-pressure fluid supply tube 64 and the fluid supply tube 70 from contacting a roller pump (not explicitly shown) provided with the drive unit 12, such as located in the carriage assembly 22 or adjacent thereto.

FIG. 3 is a partially exploded side view of the elements of FIG. 2 illustrating the relationship of the pump 56, the bubble trap 60, the connection manifold assembly 62, and the fixture 140. Also shown is the vertically oriented tubular manifold 148 secured to the bracket 120. The effluent outlet port 124 may be connected to and in fluid communication with the lower interior of the tubular manifold 148. The effluent return port 126 may be connected to and in fluid communication with the upper interior of the tubular manifold 148. Also connecting to the tubular manifold 148 is a horizontally aligned passage port 150 and associated connector 132, each opposing the effluent return port 126. The passage port 150 may accommodate the high-pressure fluid supply tube 64 which extends distally through the lumen (not explicitly shown) of the passage port 150, the connector 132, the upper region of the tubular manifold 148, the effluent return port 126, the connector 142, the connection tube 144, and into and through the effluent return tube 66 in coaxial fashion to connect to the thrombectomy catheter 58 (see, for example, FIG. 1). The proximal end of the high-pressure fluid supply tube 64 includes a high-pressure fitting 152 located near the proximal end of the high-pressure fluid supply tube 64 to facilitate connection of the high-pressure fluid supply tube 64 in fluid communication with the interior of the pump 56. The proximal end of the high-pressure fluid supply tube 64, which is the inlet to the high-pressure fluid supply tube 64, may include a plurality of very small holes (not shown) comprising a filter at the proximal end thereof. The connector 134, which may have internal and/or external threads, may be aligned over and about the high-pressure fluid supply tube 64 distal to the high-pressure fitting 152 and threadingly engage a threaded connection port 154 extending horizontally from the upper portion 110 of the base 109 of the pump 56. The connector 134 may be rotated to threadably engage the high-pressure fitting 152 with corresponding mating threaded structure provided with the pump 56. A connector 132 may be utilized to engage the externally threaded end of the connector 134 to secure the connector 134, and thus the pump 56, to the connection manifold assembly 62 and to provide for fixation of the bubble trap 60 to the pump 56. In addition, direct connection and fluid communication between the pump 56 and the bubble trap 60 may be provided by a horizontally oriented pump fluid inlet port 156 which engages a corresponding receptor port 158 and seal 159 interior to one end of the bubble trap 60. The fluid inlet port 122 located on the bracket 120 may extend behind the tubular manifold 148 to communicate with the interior of the bubble trap 60 for fluid (e.g., saline) debubbling, whereby unpressurized fluid (e.g., saline) is made available for use by the pump 56.

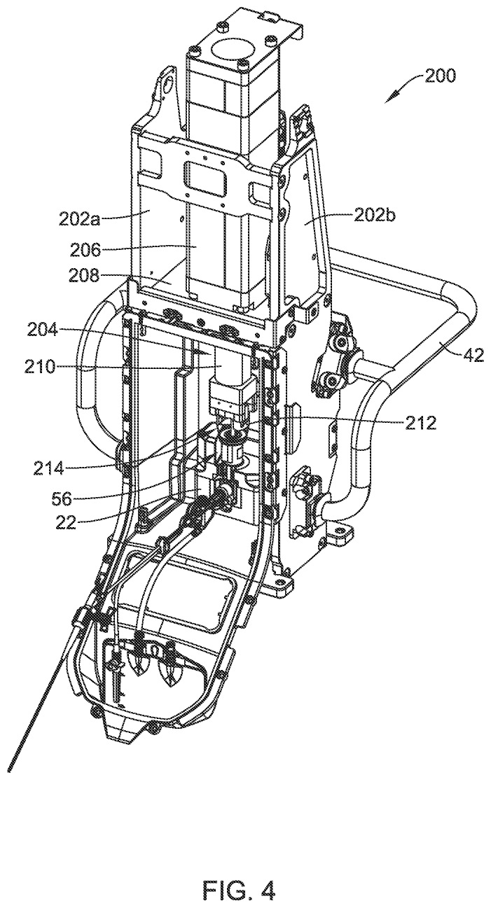

FIG. 4 is a perspective view of an upper section 200 of the drive unit 12 where the panels 16a-16n and other exterior components have been removed to reveal other components residing in the drive unit 12. The internal structure of the drive unit 12 may include two or more support structures 202a-b which serve as a mount for various components. A vertically oriented reciprocating linear actuator assembly 204 is shown extending between the intermediate portions of the support structures 202a and 202b in the 25 upper region of the drive unit 12. The reciprocating linear actuator assembly 204 may be in vertical alignment with the carriage assembly 22 for subsequent automatic engagement with the pump 56 of the pump/catheter assembly 14. The pump 56 may be aligned within and captured by components of the carriage assembly 22 with the linear actuator assembly 204 in alignment to specific regions of the carriage assembly 22 and to the pump 56.

A reciprocating linear actuator 206 may be secured to an upper surface of a mounting plate 208 and may include an actuator shaft 210 freely extending through (e.g., from the upper surface to and below of a lower surface) of the mounting plate 208 and a cylindrically-shaped pump connector 212 may be secured to the bottom of the actuator shaft 210. Downward actuation of the actuator shaft 210 may cause automatic and secure overhead snap engagement of the pump connector 212 with the pump piston head 116 of the pump 56 for subsequent reciprocating operation of the pump 56. Disengagement of the pump connector 212 from the piston pump head 116 may be occur in response to activation of a release mechanism. The release mechanism may be activated via direct user interaction with the release mechanism or via issuing a command via the user interface 32. When the release mechanism is activated hooks of the pump connector 212 may disengage from the pump piston head 116. The reciprocating linear actuator assembly 204 may then axially displace the pump connector 212 to allow the pump 56 to be pulled vertically from the carriage assembly 22.

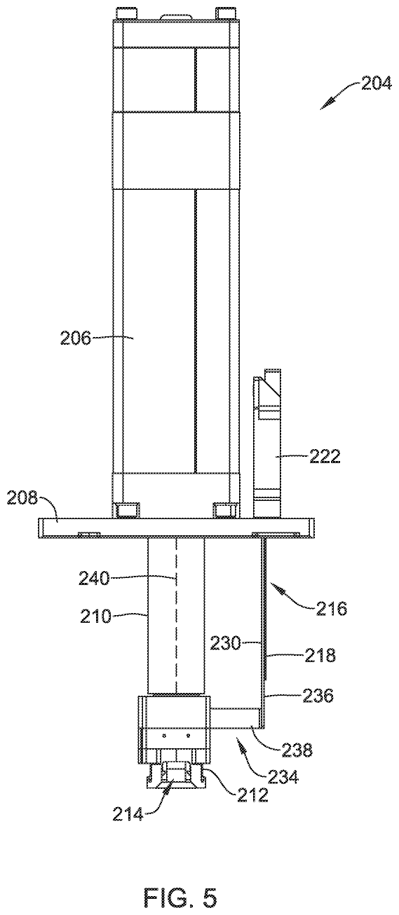

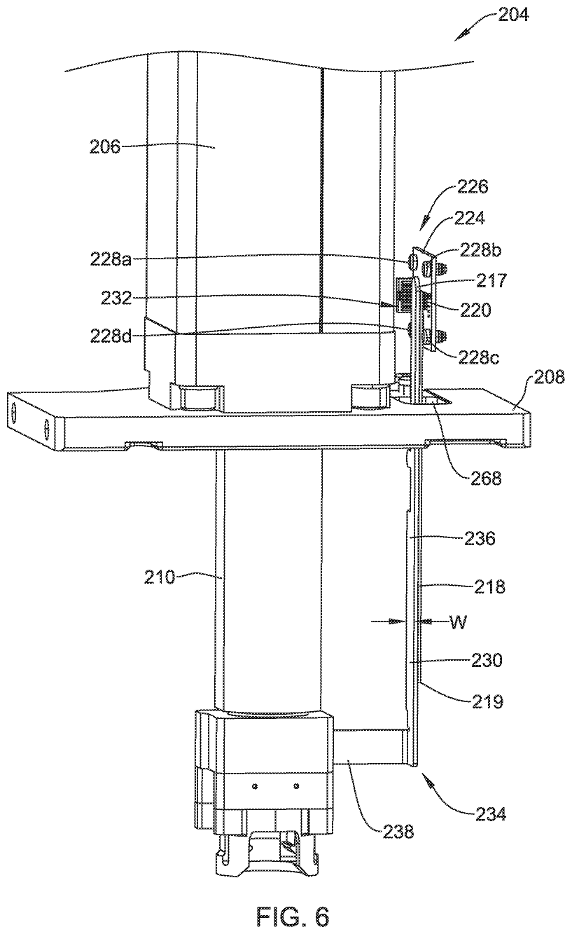

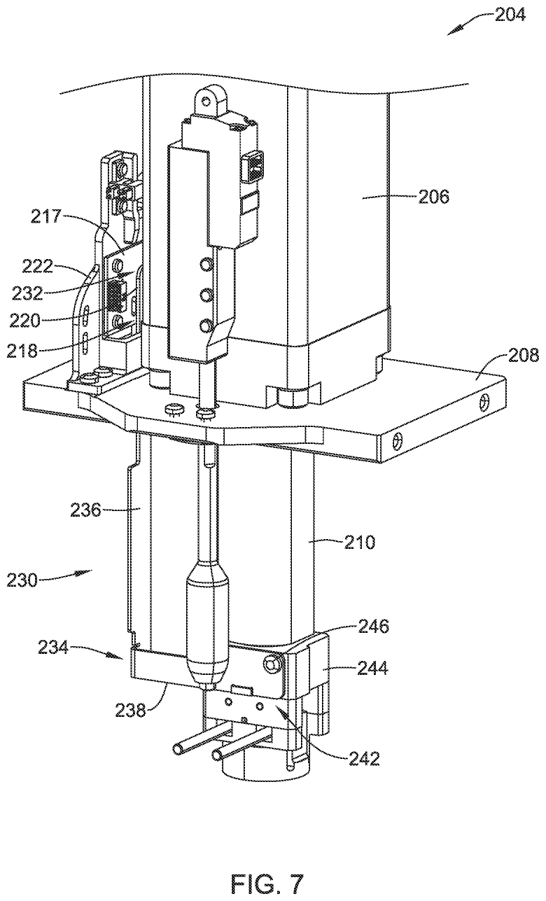

The reciprocating linear actuator assembly 204 may include a stroke length detection assembly 216 configured to determine and/or measure a relative position of the actuator shaft 210. A stroke length detection assembly 216 for stroke length detection and/or actuator position detection is shown and described with respect to FIGS. 5-7 where FIG. 5 is a front view of the illustrative reciprocating linear actuator assembly 204, FIG. 6 is an enlarged partial front perspective view of the reciprocating linear actuator assembly 204 with a mounting bracket 222 removed, and FIG. 7 is an enlarged partial rear perspective view of the reciprocating linear actuator assembly 204. In contrast to a system which utilizes fixed discrete position sensors to track the position and/or movement of the actuator shaft 210, the present stroke length detection assembly 216 may provide real-time monitoring of the position of the actuator shaft 210 for a variety of pump stroke lengths. This may allow for flexibility to use pump/catheter assemblies 14 having different pump stroke lengths or travel needed to meet different procedure requirements with a same drive unit 12. Generally, the stroke length detection assembly 216 may include a magnetic strip 218, a magnetic sensor 220, and a bracket 230.

The magnetic sensor 220 may be mounted to a printed circuit board 224 which in turn is mounted within and/or to a housing 222 or frame secured to the mounting plate 208. In some cases, the printed circuit board 224 may be coupled to the housing 222 via one or more fixation members 228a-d. The fixation members 228a-d may include, but are not limited to bolts, screws, set screws, pins, etc. The housing 222 has been removed in FIG. 6 to more particularly illustrate the arrangement of the printed circuit board 224 and the stroke length detection assembly 216. In some examples, the magnetic sensor 220 may be soldered to the printed circuit board 224. It is contemplated that the magnetic sensor 220 may be an integrated circuit such as, but not limited to, an AS5311 IC, manufactured by ams-OSRAM AG (Premstaetten, Austria). However, other linear sensors may be used, as desired. The magnetic sensor 220 may be a system-on-a-chip (SoC) that includes integrated Hall elements, analog front end and digital signal processing on a single chip. The printed circuit board 224 may be a custom printed circuit board which also contains supporting passive components and connectors. Together the magnetic sensor 220 and the printed circuit board 224 may form a printed circuit assembly 226.

The magnetic strip 218 may be secured to a portion of the bracket 230 which is coupled to the actuator shaft 210 and moveable therewith. The bracket 230 may extend from a first end region 232 adjacent to the printed circuit assembly 226 to a second end region 234 positioned between the actuator shaft 210 and the pump connector 212. The bracket 230 may have a generally “L” shaped configuration and include a first portion 236 extending generally parallel to a longitudinal axis 240 of the actuator shaft 210 and a second portion 238 extending generally orthogonal to the longitudinal axis 240 of the actuator shaft 210. However, the bracket 230 may take other configurations as desired. For example, the second portion 238 may extend at a non-orthogonal angle to the longitudinal axis 240 of the actuator shaft 210. The first portion 236 may be laterally spaced from the actuator shaft 210. The second portion 238 of the bracket 230 may be affixed or coupled to the actuator shaft 210 such that as the actuator shaft 210 is linearly displaced along the longitudinal axis 240 thereof the bracket 230 is also linearly displaced. For example, a free end 242 of the second portion 238 (e.g., the end not coupled to the first portion 236 of the bracket 230) may be coupled to a lead screw piston 244 of the actuator shaft 210 via one or more fixation members 246. The fixation members 246 may include, but are not limited to bolts, screws, set screws, pins, etc. In some cases, the fixation members 246 may be configured to adjust a distance between the magnetic strip 218 and the magnetic sensor 220. For example, the position of the bracket 230 may be adjusted to bring the magnetic strip 218 closer to the magnetic sensor 220 or to move the magnetic strip 218 further from the magnetic sensor 220. However, this is not required. In some examples, the tolerances of the drive unit 12 and components thereof may be tight enough that the position of the bracket 230 does not need to be adjusted to place the magnetic strip 218 in a desired position relative to the magnetic sensor 220. It is contemplated that the magnetic strip 218 may be positioned within a predetermined distance of the magnetic sensor 220. In some embodiments, the magnetic strip 218 may be positioned at a distance of approximately 0.6 millimeters (mm) or less from the magnetic sensor 220. However, in some embodiments, the distance may be greater than 0.6 mm. The fixation members 246 may be secured or tightened after the distance between the magnetic strip 218 and the magnetic sensor 220 has been adjusted (if adjustable) to ensure that the distance between the magnetic strip 218 and the magnetic sensor 220 is fixed or locked.

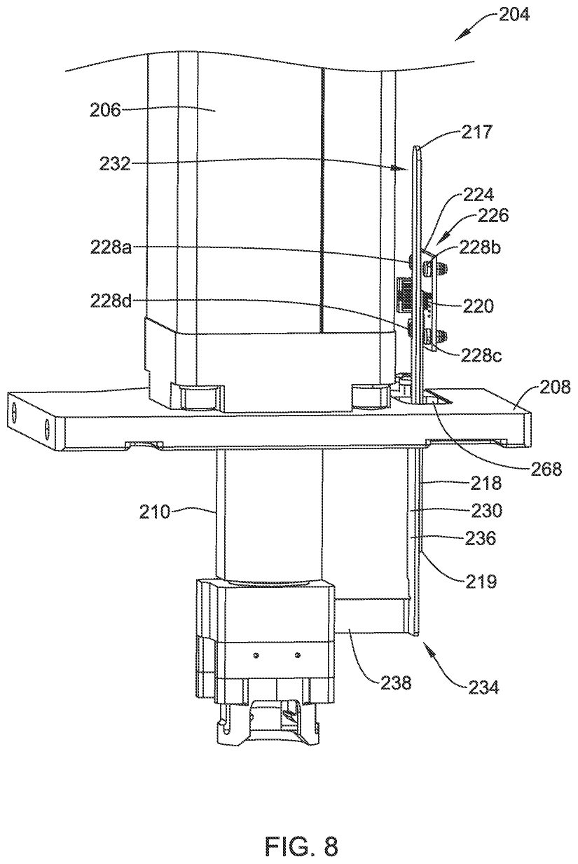

The first end region 232 of the bracket 230 may extend through an opening 268 in the mounting plate 208 such that the first end region 232 is adjacent to the magnetic sensor 220 when the actuator shaft 210 is at the bottom of the stroke, as shown in FIGS. 5-7. As the actuator shaft 210 moves upwards, the bracket 230 moves up with the actuator shaft 210. FIG. 8 is an enlarged partial front perspective view of the reciprocating linear actuator assembly 204 with a mounting bracket 222 removed and the actuator shaft 210 axially displaced or raised. As can be seen in FIG. 8, as the actuator shaft 210 is raised, the first end region 232 of the bracket 230 shifts upwards as well. As the bracket 230 moves, the magnetic strip 218 also moves.

The magnetic strip 218 may be secured to a surface of the first portion 236 of the bracket 230. For example, the magnetic strip 218 may be secured to the surface of the first portion 236 that faces towards the magnetic sensor 220. For example, the magnetic strip 218 may be positioned between the first portion of the bracket 230 and the magnetic sensor 220. In the illustrated embodiment, the magnetic strip 218 is mounted on a surface which faces away from the actuator shaft 210. However, this is not required. It is contemplated that the placement of the magnetic strip 218 may depend on the location of the magnetic sensor 220. The magnetic strip 218 may be secured to the bracket 230, using, for example, an adhesive strip. However, other means for securing the magnetic strip 218 to the bracket 230 may be used, as desired. The magnetic strip 218 may have a length extending from a first end 217 to a second end 219. Further, the magnetic strip 218 may have a width that is approximately equal to or less than a width W of the first portion 236 of the bracket 230. In some cases, the magnetic strip 218 may extend along an entirety of a length of the first portion 236 of the bracket 230. In other embodiments, the magnetic strip 218 may extend along less than an entirety of the length of the first portion 236 of the bracket 230. It is contemplated that the magnetic strip 218 may have a length that allows the magnetic strip to be adjacent to the magnetic sensor 220 for an entirety of the stroke length of the actuator shaft 210. For example, the magnetic strip 218 may have a length that is approximately equal to or greater than a stroke length of the actuator shaft 210

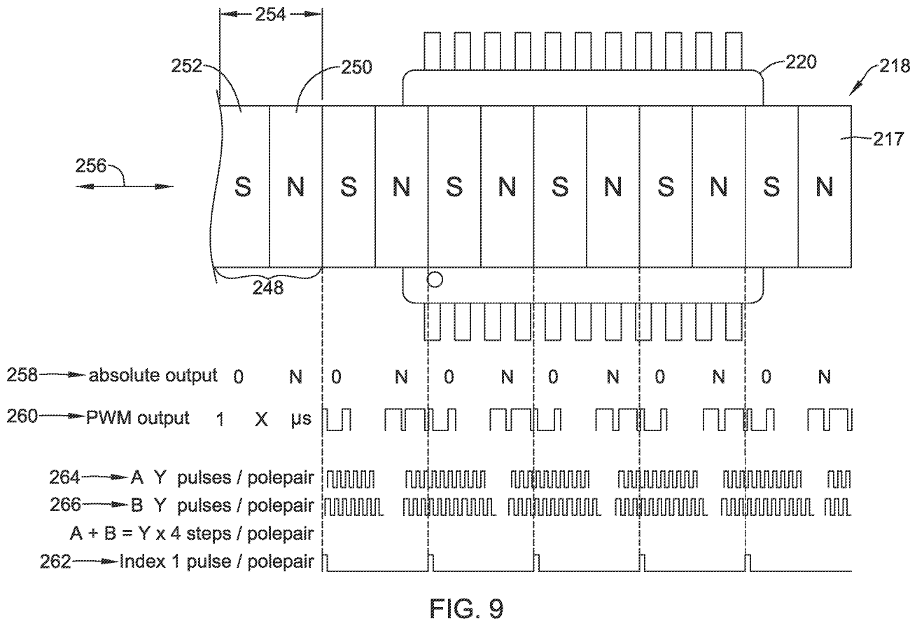

FIG. 9 illustrates a front view of a portion of the magnetic strip 218 adjacent to the magnetic sensor 220 and a plurality of electrical outputs or signals output from the magnetic sensor 220. The magnetic strip 218 may include a plurality of north and south pole pairs 248. The portion of the magnetic strip 218 illustrated in FIG. 9 is not necessarily to scale. Further, for brevity and case of understanding not every north and south pole pair 248 has been identified with a reference number. The north and south pole pairs 248 may each include a north pole 250 and a south pole 252 extending across a width of the magnetic strip 218. The north and south pole pairs 248 may repeat continuously along a length of the magnetic strip 218 (e.g., from the first end 217 to the second end 219). Each north and south pole pair 248 may have a length 254 of approximately 2 millimeters (mm) such that each pole 250, 252 has a length of approximately 1 mm. However, each north and south pole pair 248 may have a length 254 of less than 2 mm or greater than 2 mm, as desired.

As described above, the magnetic strip 218 is positioned on the bracket 230 such that the magnetic strip 218 moves up and down vertically (e.g., parallel to the longitudinal axis 240 of the actuator shaft 210) next to the magnetic sensor 220 as the actuator shaft 210 reciprocates to actuate the pump 58. The direction of travel of the magnetic strip 218 is illustrated at arrow 256. When the magnetic strip 218 and the magnetic sensor 220 are assembled with the drive unit 12, the direction of travel 256 of the magnetic strip 218 is parallel to the longitudinal axis 240 of the actuator shaft 210. As the bracket 230 is coupled to the lead screw piston 244 of the actuator shaft 210, the magnetic strip 218 moves in direct proportion to the distance the actuator shaft 210 moves. As the magnetic strip 218 moves (as shown at arrow 256), the magnetic sensor 220 may output a plurality of data outputs or signals related to a position of the magnetic strip 218. For example, the magnetic sensor 220 may output an absolute output 258 which includes a count from 0 to a predetermined maximum N for each north and south pole pair 248 and repeats for each north and south pole pair 248. For example, with each new north and south pole pair 248, the count begins at 0. In some examples, the predetermined maximum absolute output count may be 4095. However, this is not required. The predetermined maximum may be determined by the type of magnetic sensor 220 and/or a length 254 of the north and south pole pair 248. It is contemplated that the absolute output may provide an indication of the position of the magnetic strip 218 within one particular north and south pole pair 248. The magnetic sensor 220 may also output a pulse-width modulation (PWM) output 260. The PWM may begin with a first pulse width (measure in microseconds (μs)) and may increase the pulse width with every step (the step having a predetermined distance) such that the PWM reaches a maximum pulse width X at the end of north and south pole pair 248. In some examples, the pulse width may increase with every step of 0.488 micrometers (μm) and may reach a maximum pulse width of 4097 μs at the end of north and south pole pair 248. An index pulse 262 may be generated once for north and south pole pair 248. Finally, the magnetic sensor 220 may also include an “A” output 264 and a “B” output 266. The A output 264 and a B output 266 may be phase shifted by about 90 electrical degrees. Thus, the number of edges for the A and B outputs 264, 266 may be the number of incremental pulses Y per north and south pole pair 248 multiplied by 4 (for example, each of the “A” output 264 and a “B” output 266 may include a leading edge and a trailing edge for each incremental pulse). In an illustrative embodiment, 256 incremental pulses may be generated at each of the A output 264 and the B output 266 for every north and south pole pair 248. Thus, a system with 256 incremental pulses may include 1024 edges per north and south pole pair 248. The incremental pulses may be repeated with each north and south pole pair 248 as the magnetic strip 218 is actuated next to the magnetic sensor 220. Each incremental pulse may represent an incremental distance moved by the magnetic strip 218 (and thus the actuator shaft 210). In some cases, each incremental pulse may be correlated to a distance in the micrometer range. It is contemplated that which of the A output 264 and the B output 266 is the leading edge and which is the trailing edge of each incremental pulse may depend on the direction of travel of the magnetic strip 218.

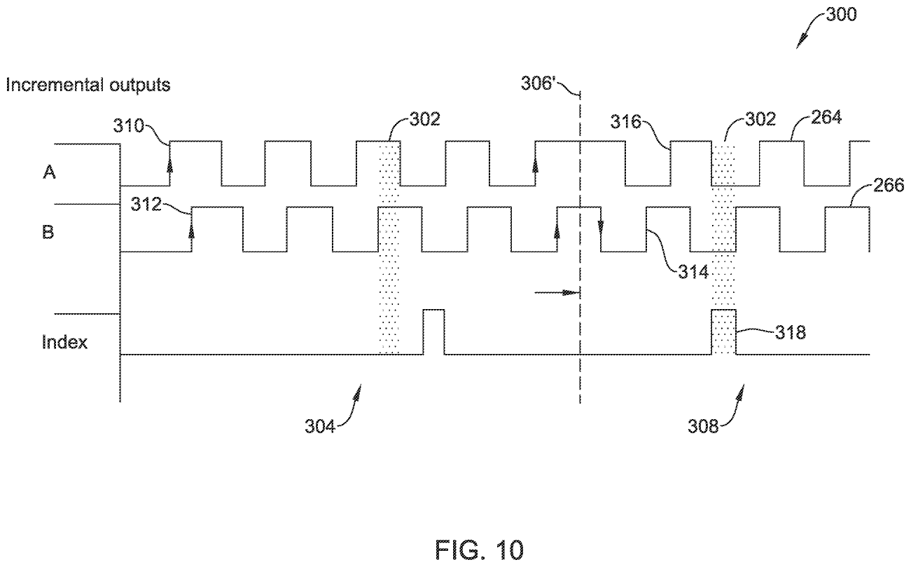

FIG. 10 is a schematic graph 300 illustrating the A and B incremental pulse outputs 264, 266 as the magnetic strip 218 moves relative to the magnetic sensor 220. Generally, the field programmable gate array (FPGA) logic in the system-on-a chip (e.g., the magnetic sensor 220) may read the state of the A incremental pulse output 264 and the B incremental pulse output 266. If the A signal 264 rises from 0 to 1 while the B signal 266 is 0 then the actuator shaft 210 has incrementally moved in one direction. Conversely, if the B signal 266 rises while the A signal 264 is at 0 the actuator shaft 210 has moved in the opposite direction. The A and B incremental pulse outputs 264, 266 may both rise and fall for each incremental step of the linear encoder. The FPGA may keep a running count of every rising edge and depending on if the A incremental pulse output 264 or B incremental pulse output 266 rises first this count either increments or decrements. This count may then correspond to the actuator shaft 210 position as the FPGA may track the actuator shaft 210 from when the count is “zeroed” by the firmware. Therefore, this may be a relative measurement of the actuator motion. The FPGA may also check upper and lower travel limits of the actuator shaft 210 defined by the firmware and may flag or identify when the actuator shaft 210 has traveled outside of these limits. In some cases, an alert may be displayed on the user interface when the actuator shaft 210 travels outside of the predetermined upper and/or lower limits. The upper and/or lower limits may be defined dynamically by firmware through an FPGA register and can be changed or adjusted. It is contemplated that the magnetic sensor 220 may be in communication with the user interface 32 to allow the user to view information related to position of the actuator shaft 210 and/or stroke length.

A mechanical zero position is shown at 302. The mechanical zero position 302 may be generated by zeroing the magnetic sensor reading location in the software. In some cases, the zeroing step may be performed at the user interface 32. It is contemplated that the mechanical zero position 302 may not be the same each time the drive unit 12 is utilized. The mechanical zero position 302 may be at any configuration of the reciprocating linear actuator assembly 204 at the time of zeroing. For example, the stroke length detection assembly 216 may be at the lowest position, the highest position, or any position in between the lowest and highest positions at the time of zeroing. The A and B incremental pulse outputs 264, 266 may be relative to the mechanical zero position 302 and not an absolute value corresponding to a specific point on the magnetic strip 218. Any travel of the actuator shaft 210 (and thus the magnetic strip 218) after the mechanical zero position 302 has been determined will be measured and processed by the magnetic sensor 220. This data may be output to the user interface 32

The graph 300 illustrates an index count 318. As noted above, the index count may represent each north and south pole pair 248. A first portion 304 of the graph 300 may illustrate the A and B incremental pulse outputs 264, 266 as the magnetic strip 218 moves up to down and a second portion 308 of the graph 300 may illustrate the A and B incremental pulse outputs 264, 266 as the magnetic strip 218 moves down to up. For brevity and ease of understanding, the illustrated graph 300 may not show each incremental pulse for each north and south pole pair 248. The change in direction of travel of the magnetic strip 218 is shown at 306. When the magnetic strip 218 is moving up to down, the A incremental pulse output 264 may lead or occur before the B incremental pulse output 266. For example, a leading edge 310 of the A incremental pulse output 264 occurs about 90 electrical degrees before a leading edge 312 of the B incremental pulse output 266 when the magnetic strip 218 is moving up to down. When the magnetic strip 218 is moving down to up, the B incremental pulse output 266 may lead or occur before the A incremental pulse output 264. For example, a leading edge 314 of the B incremental pulse output 266 occurs about 90 electrical degrees before a leading edge 316 of the A incremental pulse output 264 when the magnetic strip 218 is moving down to up. Thus, the magnetic sensor 220 may determine a direction of travel of the magnetic strip 218 (and thus the actuator shaft 210) based on which of the A or B incremental pulse outputs 264, 266 is output first. In the illustrated embodiment of FIG. 10, the magnetic strip 218 moves in the up to down direction for a period of time, as shown at 304. As the magnetic strip 218 moves, the mechanical zero position 302 is passed. At 306, the direction of travel of the magnetic strip 218 is reversed and the magnetic strip 218 moves in the down to up direction. Once again, the mechanical zero position 302 is passed. As can be seen in FIG. 10, a same number of pulses occur between the mechanical zero position 302 and the direction reversal 306 regardless of a direction of travel.

The magnetic sensor 220 may further count the number of incremental pulse outputs to determine a distance of travel. For example, every incremental pulse output may be an incremental movement of the magnetic strip 218 (e.g., the incremental movement may be in the micrometer range). The magnetic sensor 220 may add to the count when the magnetic strip 218 is traveling in a first direction and may subtract from the count when the magnetic strip 218 is traveling in a second direction opposite the first direction to determine a relative distance from the mechanical zero position 302. It is further contemplated that the distance traveled may also be used to determine the speed of travel of the actuator shaft 210. In some examples, the actuator shaft 210 may have a downstroke speed in the range of about 6.72 inches per second (17.07 centimeters per second) and an upstroke speed in the range of about 12 inches per second (30.5 centimeters per second). The determined travel distance and speed may be used to check for proper operation of travel distance and velocity.

The materials that can be used for the various components of the thrombectomy catheter, pump/catheter assembly, and/or other devices disclosed herein may include those commonly associated with medical devices. For simplicity purposes, the following discussion makes reference to the pump/catheter assembly and its related components. However, this is not intended to limit the devices and methods described herein, as the discussion may be applied to other similar devices, tubular members and/or components of tubular members or devices disclosed herein.

The various components of the devices/systems disclosed herein may include a metal, metal alloy, polymer (some examples of which are disclosed herein), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-clastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-NR® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In at least some embodiments, portions or all of the pump/catheter assembly and its related components may be doped with, made of, or otherwise include a radiopaque material. Radiopaque materials are understood to be materials capable of producing a relatively bright image on a fluoroscopy screen or another imaging technique during a medical procedure. This relatively bright image aids the user of the pump/catheter assembly and its related components in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the pump/catheter assembly and its related components to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A drive unit for a thrombectomy system, the drive unit comprising:
one or more panels enclosing an internal structure of the drive unit;
a vertically oriented reciprocating linear actuator assembly extending between one or more support structures in an upper region of the drive unit, the vertically oriented reciprocating linear actuator assembly including an actuator shaft;
a bracket coupled to the actuator shaft and moveable therewith, the bracket including a first portion extending generally parallel to a longitudinal axis of the actuator shaft;
a magnetic strip affixed to a surface of the first portion of the bracket; and
a magnetic sensor positioned adjacent to a first end region of the bracket;
wherein the magnetic sensor is configured to determine a relative position of the actuator shaft.

2. The drive unit of claim 1, wherein the magnetic sensor is secured within a housing secured to an upper surface of a mounting plate.

3. The drive unit claim 1, wherein the magnetic sensor is mounted to a printed circuit board.

4. The drive unit of claim 1, wherein the first portion of the bracket is configured to extend through an opening in a mounting plate.

5. The drive unit of claim 1, wherein the magnetic strip is positioned between the bracket and the magnetic sensor.

6. The drive unit of claim 1, wherein the magnetic strip includes a plurality of north and south pole pairs.

7. The drive unit of claim 6, wherein the north and south pole pairs extend along a length of the magnetic strip.

8. The drive unit of claim 1, wherein the magnetic sensor comprises a system-on-a-chip.

9. The drive unit of claim 1, wherein the magnetic sensor is configured to output an “A” signal and a “B” signal.

10. The drive unit of claim 9, wherein if the “A” signal rises from 0 to 1 while the “B” signal is 0, the actuator shaft is moving in a first direction.

11. The drive unit of claim 10, wherein if the “B” signal rises from 0 to 1 while the “A” signal is 0, the actuator shaft is moving in a second direction opposite the first direction.

12. The drive unit of claim 11, wherein if the actuator shaft is moving in the first direction, the magnetic sensor increments a count of rising edges and if the actuator shaft is moving in the second direction, the magnetic sensor decrements the count of rising edges.

13. The drive unit of claim 12, wherein the count of rising edges is directly correlated to distance traveled by the actuator shaft.

14. The drive unit of claim 1, wherein the magnetic strip has a length approximately equal to or greater than a stroke length of the actuator shaft.

15. The drive unit of claim 1, wherein the magnetic strip is linearly displaced in direct proportion to linear movement of the actuator shaft.

16. A drive unit for a thrombectomy system, the drive unit comprising:
one or more panels enclosing an internal structure of the drive unit;
a vertically oriented reciprocating linear actuator assembly extending between one or more support structures in an upper region of the drive unit, the vertically oriented reciprocating linear actuator assembly including an actuator shaft;
a housing;
a magnetic sensor secured within the housing;
a bracket coupled to the actuator shaft and moveable direct proportion therewith, the bracket including a first portion extending generally parallel to a longitudinal axis of the actuator shaft; and
a magnetic strip including a plurality of north and south pole pairs affixed to a surface of the first portion of the bracket between the bracket and the magnetic sensor;
wherein the magnetic sensor is configured to increment or decrement an incremental pulse count as the magnetic strip moves with the actuator shaft and to determine a distance of travel of the actuator shaft relative to a mechanical zero position based on the incremental pulse count.

17. The drive unit of claim 16, wherein the magnetic sensor is configured to output an “A” incremental pulse signal and a “B” incremental pulse signal.

18. The drive unit of claim 16, wherein if the actuator shaft is moving in a first direction, the magnetic sensor increments the incremental pulse count and if the actuator shaft is moving in a second direction opposite the first direction, the magnetic sensor decrements the incremental pulse count.

19. The drive unit of claim 16, wherein the magnetic strip is spaced 0.6 millimeters or less from magnetic sensor.

20. A method for determining a distance of travel of an actuator shaft of a drive unit for a thrombectomy system, the method comprising:
identifying a mechanical zero position of an actuator shaft relative to a magnetic strip;
incrementing an incremental pulse count as the magnetic strip moves with the actuator shaft in a first direction;
decrementing the incremental pulse count as the magnetic strip moves with the actuator shaft in a second direction opposite the first direction;
determining a distance and direction traveled by the actuator shaft based on the incremental pulse count; and
outputting the distance and/or direction to a user interface.
